(19) United States
(12) Patent Application Publication
(10) Pub. No.: US 2026/0198949 A1
(43) Pub. Date: Jul. 16, 2026

(54) MECHANICAL THROMBECTOMY DEVICES AND METHODS OF USE

(71) Applicant: Purdue Research Foundation, West Lafayette, IN (US)

(72) Inventors: Hyowon Lee, West Lafayette, IN (US); Angel G. Enriquez, Albany, NY (US); Daniel Sahlein, Indianapolis, IN (US); Peter Kim Nelson, Forest Hills, NY (US)

(21) Appl. No.: 19/154,202

(22) PCT Filed: Feb. 6, 2024
(86) PCT No.: PCT/US2024/014626

§ 371 Date: Aug. 6, 2025

Related U.S. Application Data
(60) Provisional application No. 63/483,453, filed on Feb. 6, 2023.

(51) Int. Cl. A61B 17/221; A61B 17/00; A61B 17/3207

(52) U.S. Cl. CPC A61B 17/221; A61B 17/320758; A61B 2017/00526; A61B 2017/2215; A61B 2017/320775; A61B 2217/005

(57) ABSTRACT

Mechanical thrombectomy devices adapted for removing an occluding blood clot in a patient, and methods of manufacturing and using mechanical thrombectomy devices. Such a mechanical thrombectomy device includes a bore defining an interior passage of an aspiration catheter, and microstructures extending radially into the interior passage. The microstructures are adapted to engage a clot drawn into the aspiration catheter.

### BACKGROUND OF THE INVENTION

The invention generally relates to devices and methods for treating strokes. The invention relates specifically to devices and methods relating to mechanical thrombectomy.

Stroke is the second leading cause of death worldwide. Large vessel occlusion (LVO), the obstruction of large cerebral arteries by clots, accounts for 40% of stroke cases and effective treatment of such a condition is extremely time-sensitive in order to reduce brain damage. Specifically, it is imperative to restore blood flow, known as hemodynamic flow, by reopening occluded vessels and providing reperfusion to salvage damaged or threatened tissues. In order to do so, the occluding clot must be dissolved or removed effectively and quickly.

Currently the use of chemical agents to break up clots, a process known as thrombolytic therapy, is the first line of treatment when a patient suffers a stroke. However, this treatment loses effectiveness in cases of LVO and worsens as the region of occlusion extends to the terminal internal carotid artery, the artery in the neck which provides blood flow to the anterior portion of the brain. Mechanical thrombectomy, using physical interaction through aspiration or stent-retrievers to restore blood flow, has emerged as a standard of care for LVO. Mechanical thrombectomy provides significant advantages in the timely removal of occluding clots and the effective reperfusion of threatened tissue.

A variety of strategies for mechanical thrombectomy have emerged. Most techniques involve the use of a stent retriever, an aspiration catheter (AC), or both. In both cases, a catheter is inserted into the patient's vascular system, often at the groin, and mechanically guided through the vascular system to the location of the clot. In the case of a stent retriever, the occluding clot is crossed with a microcatheter, the retrievable stent is unsheathed from the microcatheter and expands, allowing struts to embed themselves in the clots and expand the arterial walls, thereby providing reperfusion. The stent is then retrieved into the catheter, bringing the clot with it.

ACs are most commonly employed using a direct aspiration first-pass technique, whereby the AC, attached to a device which provides negative pressure and therefore provides a vacuum effect at the end of the AC, is brought over a microcatheter and microwire to the face of the clot. The microcatheter and microwire are removed, the clot is subjected to vacuum aspiration, and the clot engages with the AC and is dissolved or effectively removed. Once the clot is sufficiently destroyed or removed, as observed by blood being vacuumed through the catheter indicating removal of the occlusion, or some other means of observation known to those skilled in the art, the AC is removed.

ACs are an attractive device for treating LVO; studies show that ACs provide faster reperfusion, lower rates of distant emboli caused by debris from the original clot, greater cost effectiveness than stent retrieval. Advances in methods of mechanical thrombectomy have been directed at more effective clot and clot debris removal on the first pass of such catheters through a blood vessel, thereby reducing time required for treatment and the risk associated with guiding a catheter through the entirety of the vascular system network. ACs require increased force with which a clot is captured in order to improve treatment, and as a result improvements in AC devices trend towards providing larger inner diameters in order to provide greater force. However, diameters of existing ACs have been limited by blood vessel diameters, particularly when clots occur in distant locations in the vascular network.

There is an ongoing need to improve the recanalization rates and first pass effect of ACs in distal emboli where larger bores (e.g., greater than about 0.08 inch (about 2 mm)) are not feasible. Stent retrievers used in distal blood vessels in the M2 or M3 segments of the middle cerebral artery (MCA) risk injuring blood vessels and require increased intervention from the surgeon, raising surgery time and costs. Though ACs are relatively simple and cost-effective for procedures performed in distal blood vessels, increased forces with which clots can be captured are required to improve patient outcomes and avoid embolization.

It would be advantageous to provide a device or method capable of improving the effectiveness of AC devices in mechanical thrombectomy without increasing the diameter of the AC itself.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, devices and methods relating to mechanical thrombectomy.

According to a nonlimiting aspect of the invention, a mechanical thrombectomy device includes a bore defining an interior passage of an aspiration catheter, and microstructures extending radially into the interior passage. The microstructures are adapted to engage a clot drawn into the aspiration catheter.

In some arrangements, the microstructures may be spear-shaped, or 3D-printed triangular-shaped, or some combination thereof. The microstructures are configured in such a way that they engage a targeted occluding clot, and are preferably capable of improving the force with which a clot is captured.

According to another nonlimiting aspect of the invention, a method of using a mechanical thrombectomy device as described above includes guiding the aspiration catheter to an occluding blood clot within a human vascular system and making contact between the aspiration catheter and the blood clot, drawing a vacuum within the aspiration catheter to draw the occluding clot into the bore of the catheter and engage the occluding clot with the microstructures, and then retracting the aspiration catheter, thereby retrieving the occluding clot.

In some embodiments of the aforementioned invention, variations of the method may further include the use of devices and techniques that may be utilized to place an aspiration catheter and/or remove an occluding clot.

These and other aspects, arrangements, features, and/or technical effects will become apparent upon detailed inspection of the figures and the following description.

### BRIEF DESCRIPTION OF THE DRAWINGS

FIG. 1A is an image of an unfolded structure that was fabricated by laser micromachining a NiTi sheet to comprise microstructures (less than 10 micrometers). The white bar represents a length of 0.5 millimeter (mm) and is included for scale.

FIG. 1B is an image of a structure that was fabricated by 3D-printing to comprise microstructures using 2-photon laser lithography (2PLL). The white bar represents a length of 0.5 mm and is included for scale.

FIG. 1C is an image of a structure formed with an unfolded structure of a type shown in FIG. 1A and after being fixed inside the inner diameter of a Sofia 5F catheter. The white bar represents a length of 1 mm and is included for scale.

FIG. 1D is an image of a structure of a type shown in FIG. 1B after being fixed inside the inner diameter of a Sofia 5F catheter. The white bar represents a length of 1 mm and is included for scale.

FIG. 2 is image showing an axial end view of a structure of a type shown in FIG. 1B after being fixed inside the inner diameter of a silicone tube, evidencing the ability of the microstructures thereof to interrupt the inner volume of an aspiration catheter in which the structure may be placed for use as a mechanical thrombectomy device. The white bar represents a length of 1 mm and is included for scale.

### DETAILED DESCRIPTION OF THE INVENTION

The intended purpose of the following detailed description of the invention and the phraseology and terminology employed therein is to describe what is shown in the drawings, which depict and/or relate to one or more nonlimiting embodiments of the invention, and to describe certain but not all aspects of the embodiment(s) depicted in the drawings. The following detailed description also identifies certain but not all alternatives of the embodiment(s) depicted in the drawings. As nonlimiting examples, the invention encompasses additional or alternative embodiments in which one or more features or aspects shown and/or described as part of a particular embodiment could be eliminated, and also encompasses additional or alternative embodiments that combine two or more features or aspects shown and/or described as part of different embodiments. Therefore, the appended claims, and not the detailed description, are intended to particularly point out subject matter regarded to be aspects of the invention, including certain but not necessarily all of the aspects and alternatives described in the detailed description.

To facilitate the description provided below of the embodiment(s) represented in the drawings, relative terms, including but not limited to, “proximal,” “distal,” “anterior,” “posterior,” “vertical,” “horizontal,” “lateral,” “front,” “rear,” “side,” “forward,” “rearward,” “top,” “bottom,” “upper,” “lower,” “above,” “below,” “right,” “left,” etc., may be used in reference to the orientation of the handheld towel rack during its use and/or as represented in the drawings. All such relative terms are useful to describe the illustrated embodiment(s) but should not be otherwise interpreted as limiting the scope of the invention.

According to a nonlimiting aspect of the invention, mechanical thrombectomy devices are provided that are capable of being placed within a catheter, for example, an aspiration catheter (AC), and are equipped with microstructures capable of disrupting the inner volume of the catheter. Such a device, which may be referred to as a thrombectomy retrieval aspiration platform, generally comprises a tubular-shaped structure that, when fixed within the inner diameter of an aspiration catheter, its microstructures can be used to interact with a blood clot engaged by an aspiration catheter in such a manner that provides greater disintegration of or affixation to the blood clot, thereby improving the overall effectiveness of the thrombectomy.

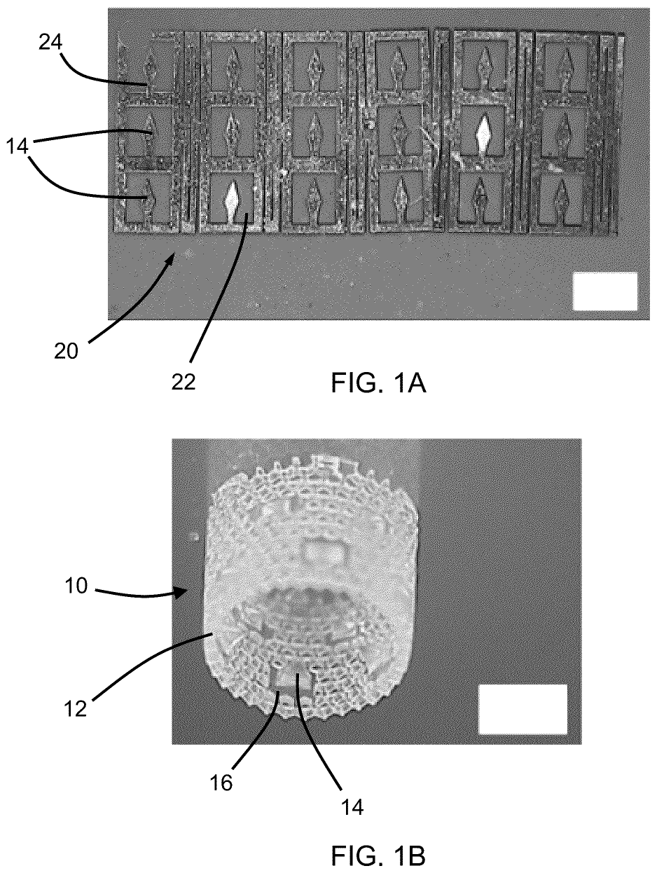

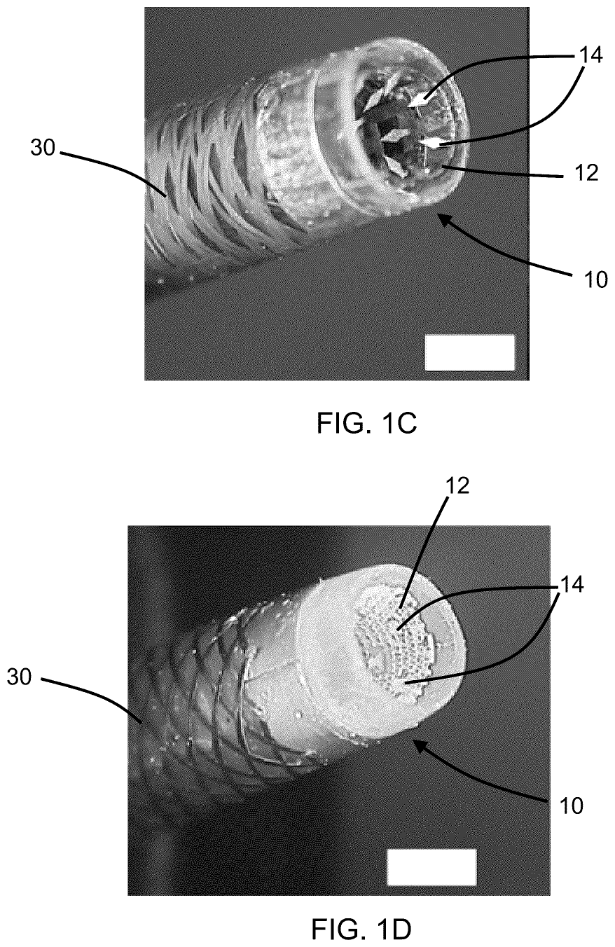

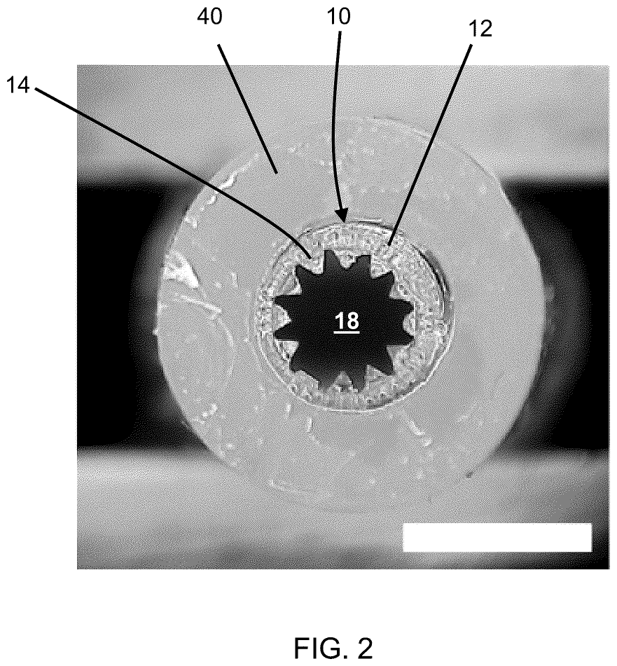

FIGS. 1A through 1D are images depicting two embodiments of mechanical thrombectomy devices 10. Each device 10 comprises a structure 12 having a generally tubular shape and microstructures 14 that project in generally radial directions into the internal passage of the structure 10. In nonlimiting embodiments of the invention, the microstructures 14 have lengths so that they extend toward, but not to, the central axis of the structure 12. The devices 10 are sized and shaped to be placed within an internal bore of a catheter 30 (e.g., FIGS. 1C and 1D), and once placed may be secured in any suitable manner, for example, with a UV-sensitive coating or adhesive.

FIG. 1A depicts a flat micromachined structure 20 that, when rolled to attain a tubular shape, forms the device 10 represented in FIG. 1C as being affixed to and within the bore of a catheter 30, and FIG. 1D shows the device 10 of FIG. 1B affixed to and within the bore of a catheter 30. The structure 20 depicted in FIG. 1A may be cut from various suitable materials, preferably a sheet of a “shape memory” nickel-titanium (NiTi) alloy. The structure 20 may be fabricated by machining the NiTi sheet, for example, by laser micromachining to define the microstructures 14. In the nonlimiting embodiment of FIGS. 1A and 1C, the microstructures 14 are each roughly ten micrometers in length and machined from an NiTi sheet having a thickness of about fifty micrometers, and the microstructures 14 have spear-like shapes and are individually disposed within square-shaped windows 22 arranged in a regular pattern of columns and rows in the structure 20. The spear-like shapes of the microstructures 14 define narrowed necks 24 where the microstructures 14 adjoin a frame of its corresponding window 22, facilitating bending of the microstructures 14 out of the planes of their respective frames. In FIG. 1C, the structure 20 has been rolled (folded) and affixed within the inner diameter of a catheter 30 such that the microstructures 14 extend radially inward into the inner volume of the catheter 30, and are thereby available to interact with a blood clot that is drawn into the inner volume of the catheter 30 by aspiration/vacuum. Customized fixtures may be integrated into equipment used to roll the structure 20 such that, after being rolled, the device 10 is receivable within the bore of the catheter 30 and capable of conforming to the inner diameter of the catheter 30.

The device 10 depicted in FIGS. 1B and 1D can be fabricated by three-dimensional (3D) printing to have both the desired tubular shape of the structure 12 and its microstructures 14. According to a nonlimiting embodiment of the invention, the 3D-printed device 10 has been produced by two-photon laser lithography (2PLL). In the nonlimiting embodiment of FIGS. 1B and 1D, the structure 12 has a web-like construction that defines a pattern of apertures through the wall of the structure 12, and the microstructures 14 have triangular shapes and protrude radially inward from integral bases 16 of the structure 20 that lack apertures to provide a rigid foundation and attachment for each microstructure 14. As shown in FIG. 1D, the device 10 is affixed within the inner diameter of a catheter 30 such that its microstructures 14 extend radially inward into the inner volume of the catheter 30, and are thereby available to interact with a blood clot that is drawn into the inner volume of the catheter 30 by aspiration/vacuum.

FIG. 2 depicts another mechanical thrombectomy device 10 fabricated by 3D printing and is shown fixed within a silicone tube 40 for testing purposes. The axial end view of FIG. 2 illustrates the disrupted interior of the device 10 created by its microstructures 14, and the capability thereof engaging a blood clot drawn into the interior passage 18 of the device 10.

Mechanical thrombectomy devices 10 of types as represented by the aforementioned nonlimiting embodiments are capable of providing advantages to thrombectomy and, more broadly, the treatment of strokes, particularly those originating from LVO conditions. In addition to the benefits that the use of an aspiration catheter is capable of providing to the treatment of strokes, the devices 10 provide additional force of an aspiration catheter to capture an occluding clot, as demonstrated in laboratory testing using blood clot and vascular system analogs.

Efficacy of mechanical thrombectomy devices 10 as described above were experimentally established with tensile tests performed using blood clot analogs fabricated from a 3D-printed elastic polymer. The blood clot analogs comprised a hydrogel solution of 4% guar gum mixed in a 2:1 ratio with borax diluted in deionized water resulting in a gelatinous material with a stiffness modulus of about 0.325 MPa. The tensile tests were performed with the devices 10 placed in catheter in which a reduced pressure of 60 kPa was drawn to capture the analog.

A model for evaluated the in vitro neurovascular angle of interaction was also developed to compare the clot retrieval capabilities of the devices 10 placed in aspiration catheters inserted into the tortuous environment found in the neurovasculature. Aspiration catheters in which the devices 10 were installed were inserted through a channel that mimicked the internal diameters found in the internal carotid artery (ICA) and MI regions, as well as mimicked the angle of interaction between the distal tip of the catheter and blood clots sometimes found in this tortuous region. The channel mimicked a 70° branch to provide a more challenging interaction between the catheter and blood clot since a bend along the catheter length causes a decrease in the suction force and, therefore, would only engage the proximal end of an analog blood clot.

The investigations described above demonstrated the successful integration of the mechanical thrombectomy devices 10 inside commercial aspiration catheters. The devices 10 provided additional force with which a clot is captured due to the added penetration of the microstructures 14 along the circumference of the inner diameters of the catheters. NiTi devices 10 fabricated as depicted in FIGS. 1A and 1C achieved more than twice the tensile force compared to commercial direct aspiration catheters (0.653 N±0.016 vs. 0.302 N±0.036). Without aspiration, the devices 10 were still advantageous over the commercial catheters (0.283 N±0.024 vs. 0.13 N±0.016). 3D-printed devices 10 fabricated as depicted in FIGS. 1B and 1D achieved similar results (0.4725 N±0.08 vs. 0.167 N±0.05).

As previously noted above, though the foregoing detailed description describes certain aspects of one or more particular embodiments of the invention, alternatives could be adopted by one skilled in the art. For example, the mechanical thrombectomy devices 10 and their components could differ in appearance and construction from the embodiments described herein and shown in the drawings, functions of certain components of the mechanical thrombectomy devices 10 could be performed by components of different construction but capable of a similar (though not necessarily equivalent) function, and various materials could be used in the fabrication of the mechanical thrombectomy devices 10 and/or their components. As such, and again as was previously noted, it should be understood that the invention is not necessarily limited to any particular embodiment described herein or illustrated in the drawings.

1. A mechanical thrombectomy device comprising:
a bore defining an interior passage of an aspiration catheter; and
microstructures extending radially into the interior passage of the aspiration catheter;
wherein the microstructures are adapted to engage a clot drawn to the aspiration catheter.

2. The mechanical thrombectomy device of claim 1, wherein the microstructures project from a tubular-shaped structure.

3. The mechanical thrombectomy device of claim 1, wherein the microstructures are formed of a nickel titanium alloy.

4. The mechanical thrombectomy device of claim 2, wherein the tubular-shaped structure and the microstructures are defined by a laser-micromachined sheet of a nickel titanium alloy.

5. The mechanical thrombectomy device of claim 4, wherein the laser-micromachined sheet is rolled to form the tubular-shaped structure.

6. The mechanical thrombectomy device of claim 2, wherein the microstructures are spear-shaped, disposed in windows within the tubular-shaped structure, and attached at narrowed necks thereof to frames surrounding the windows.

7. The mechanical thrombectomy device of claim 2, wherein the tubular-shaped structure and the microstructures are an integral 3D-printed structure.

8. The mechanical thrombectomy device of claim 7, wherein the microstructures are triangular-shaped and attached to the tubular-shaped structure at bases within a wall of the tubular-shaped structure.

9. The mechanical thrombectomy device of claim 2, wherein the tubular-shaped structure is sized and shaped to be placed concentrically inside the bore of the aspiration catheter.

10. The mechanical thrombectomy device of claim 2, wherein the tubular-shaped structure is fixed to an inner diameter of the aspiration catheter by an adhesive.

11. The mechanical thrombectomy device of claim 1, wherein the microstructures extend radially inward toward but not to a central axis of the interior passage of the aspiration catheter.

12. A method of using the mechanical thrombectomy device of any of the preceding claims, the method comprising:
guiding the aspiration catheter to an occluding blood clot within a human vascular system and making contact between the aspiration catheter and the blood clot;
drawing a vacuum within the aspiration catheter to draw the occluding clot into the bore of the catheter and engage the occluding clot with the microstructures; and then
retracting the aspiration catheter, thereby retrieving the occluding clot.
